(19) United States
(12) Patent Application Publication
(10) Pub. No.: US 2024/0124838 A1
(43) Pub. Date: Apr. 18, 2024

(54) METHOD FOR SEPARATING NEURAL CREST DERIVED CELL FROM PERIPHERAL BLOOD

(71) Applicant: Hangzhou Medical College, Hangzhou (CN)

(72) Inventors: Jianlin LOU, Hangzhou (CN); Yongxin LI, Hangzhou (CN); Lingfang FENG, Hangzhou (CN); Xiaoxue GONG, Hangzhou (CN); Xiaowen DONG, Hangzhou (CN); Jiahui YAO, Hangzhou (CN); Jing HUANG, Hangzhou (CN); Shuang LIU, Hangzhou (CN); Biao XU, Hangzhou (CN); Yao QIN, Hangzhou (CN); Fan WU, Hangzhou (CN)

(21) Appl. No.: 18/379,257

(22) Filed: Oct. 12, 2023

(30) Foreign Application Priority Data
Oct. 12, 2022 (CN) 202211247274.1

(51) Int. Cl. C12N 5/0797; C12N 5/00

(52) U.S. Cl. CPC C12N 5/0623; C12N 5/0081

(57) ABSTRACT

The present disclosure provides a method for separating a neural crest derived cell from peripheral blood. In the present disclosure, a mononuclear cell is separated from the peripheral blood and then directly cultured, thereby maximizing use of a neural crest stem cell with a differentiation potential to avoid loss of the neural crest stem cell. In the method of the present disclosure, a sample to be separated is derived from the peripheral blood. The method shows less trauma and low cost. Most importantly, compared to extracting the neural crest derived cell from tissues, the neural crest derived cell extracted from the peripheral blood can be used clinically as a type of biomarker.

### CROSS REFERENCE TO RELATED APPLICATION

This patent application claims the benefit and priority of Chinese Patent Application No. 202211247274.1, filed with the China National Intellectual Property Administration on Oct. 12, 2022, the disclosure of which is incorporated by reference herein in its entirety as part of the present application.

### REFERENCE TO SEQUENCE LISTING

A computer readable XML file entitled “Sequence Listing”, that was created on Oct. 12, 2023, with a file size of about 8,303 bytes, contains the sequence listing for this application, has been filed with this application, and is hereby incorporated by reference in its entirety.

### TECHNICAL FIELD

The present disclosure belongs to the field of biotechnology, and specifically relates to a method for separating a neural crest derived cell from peripheral blood.

### BACKGROUND

Neural crest cells are a temporary group of cells that are produced by embryonic ectoderm cells during development and can produce a variety of cell types. This cell group originates from the ectoderm at an edge of the neural tube, and can migrate widely to different parts of the body after undergoing epithelial-to-mesenchymal transition (EMT), so as to form a large number of different tissues and organs (Alkobtawi M, Ray H, Barriga E H, et al. Characterization of Pax3 and Sox10 transgenic Xenopus laevis embryos as tools to study neural crest development [J]. Developmental biology, 2018, 444: S202-S208.). After migrating to a target tissue, the neural crest derived cells maintain their pluripotency in the state of neural crest stem cells, and the neural crest stem cells in the tissue further promote the production of neural crest derived derivatives. Neural crest derived cells have been found in the intestine, heart, cornea, and bone marrow of adult mammals, and the neural crest derived cells have been successfully extracted from tissues and then subjected to primary culture for experimental research on the neural crest derived cells.

Neural crest derived cells not only have stem cell characteristics but also participate in tissue repair processes. Research results have shown that when adult tissues derived from different neural crest sources respond to injury or stress, specialized cells derived from neural crest cells dedifferentiate or activate cells with neural crest stem cell characteristics remaining in the tissue. The neural crest derived cells can also repair tissues through paracrine signaling (Parfejevs V, Antunes A T, Sommer L. Injury and stress responses of adult neural crest-derived cells. Dev Biol. 2018; 444 Suppl 1: S356-S365. doi: 10.1016/j.ydbio.2018.05.011.).

A mT/mG;Wnt1-Cre mouse is obtained by crossing a Wnt1-Cre transgenic mouse with an mT/mG double-fluorescent reporter mouse. The Wnt1-Cre transgenic mouse line is widely used to study neural crest and its derivatives; while the mT/mG double-fluorescent reporter mouse has a cell membrane surface marked with a tdTomato protein before Cre recombination occurs, and then the cell membrane surface emits red fluorescence. When the mT/mG double-fluorescent reporter mouse is crossed with the Wnt1-Cre transgenic mouse, a Cre recombinase can cut out two Loxp sites located next to a tdTomato sequence under the control of a Wnt1 gene. The tdTomato sequence is connected to an enhanced green fluorescent protein (EGFP) sequence, thereby expressing an EGFP protein. As a result, the cell membrane glows green at this time. Accordingly, in the mT/mG;Wnt1-Cre mouse, neural crest derived cells are marked with the EGFP protein and then emit green fluorescence (Muzumdar M D, Tasic B, Miyamichi K, Li L, Luo L. A global double-fluorescent Cre reporter mouse. Genesis. 2007; 45(9): 593-605. doi:10.1002/dvg.20335.).

### SUMMARY

In view of the problems existing in the prior art that collecting neural crest derived cells is cumbersome and invasive, the present disclosure provides a method for separating a neural crest derived cell from peripheral blood.

The present disclosure provides a method for separating a neural crest derived cell from peripheral blood, including separating the neural crest derived cell from peripheral blood of a subject to be separated.

Preferably, the subject to be separated is an experimental animal.

Preferably, the experimental animal is selected from the group consisting of an experimental mouse, an experimental rat, and an experimental monkey.

More preferably, the method for separating a neural crest derived cell from peripheral blood specifically includes the following steps:
(1) collecting the peripheral blood and separating a buffy coat cell using a peripheral blood mononuclear cell separation medium;
(2) adding a red blood cell lysis solution to lyse a red blood cell; and
(3) centrifuging to obtain a peripheral blood mononuclear cell without red blood cells, where the neural crest derived cell is mixed in the peripheral blood mononuclear cell without red blood cells.

More preferably, the peripheral blood is collected from an orbital vein when the experimental animal is the experimental mouse.

More preferably, an mT/mG;Wnt1-Cre mouse obtained by hybridization screening of a Wnt1-Cre transgenic mouse and an mT/mG double-fluorescent reporter mouse is used when the experimental animal is the experimental mouse.

More preferably, the peripheral blood is collected to separate the neural crest derived cell after silicosis fibrosis is induced in the mT/mG;Wnt1-Cre mouse by silica.

Even more preferably, the method for separating a neural crest derived cell from peripheral blood specifically includes the following steps:
(1) collecting 1 mL of anticoagulated blood as the peripheral blood, and diluting the peripheral blood with a PBS solution at a volume ratio of 1:1;
(2) slowly adding obtained diluted peripheral blood onto a liquid surface of 3 mL of a mouse peripheral blood mononuclear cell separation medium;
(3) centrifuging at 400 g and 20° C. for 30 min;
(4) collecting at least 800 μL of the buffy coat cell and adding the PBS solution to 14 mL;
(5) centrifuging at 300 g for 10 min;
(6) discarding a resulting supernatant, adding 3 mL of the red blood cell lysis solution, mixing the remaining red blood cell evenly by gently pipetting, and lysing the red blood cell at a room temperature for 2 min;
(7) centrifuging at 400 g and 4° C. for 5 min;
(8) discarding a resulting red supernatant, and adding 10 mL of the PBS solution, and mixing a remaining cell evenly by gently pipetting;
(9) centrifuging at 400 g for 3 min; discarding a resulting supernatant;
(10) optional step: adding 10 mL of the PBS solution, and mixing a remaining cell evenly by gently pipetting; centrifuging at 400 g for 3 min; discarding a resulting supernatant;
(11) resuspending the cell in a newly configured DMEM/F12 medium and conducting cell counting;
(12) culturing the cell in vitro: changing the DMEM/F12 medium with a new medium three days after the cell is inoculated, conducting semi-quantitative medium change every 3 d to 4 d for first two weeks, conducting digestion and subculture when a cell confluence reaches 80%, and changing the medium once a week for six weeks to obtain the neural crest derived cell.

The present disclosure has the following advantages:

In the present disclosure, the neural crest derived cell separated by the method has been labeled with EGFP. Under 488-nm wavelength excitation, a cell membrane surface can emit a green fluorescent signal, making it easy to distinguish.

In the present disclosure, a mononuclear cell is separated from the peripheral blood and then directly cultured, thereby maximizing the use of a neural crest stem cell with a differentiation potential to avoid loss of the neural crest stem cell.

In the present disclosure, the method establishes a methodology for the separation and culture of neural crest derived cells from mouse peripheral blood, laying a foundation for such research.

In the method of the present disclosure, a sample to be separated is derived from the peripheral blood. The method shows less trauma and low cost. Most importantly, compared to extracting the neural crest derived cell from tissues, the neural crest derived cell extracted from the peripheral blood can be used clinically as a type of biomarker.

### BRIEF DESCRIPTION OF THE DRAWINGS

FIG. 1 shows a generation principle of the mT/mG;Wnt1-Cre transgenic mouse;

FIG. 2 shows a genotype of the mT/mG;Wnt1-Cre transgenic mouse, where a selected box represents the genotype of this mouse;

FIG. 3A to FIG. 3C show that a group of EGFP-positive cells in fibrotic lesions of the mT/mG;Wnt1-Cre transgenic mouse exhibit characteristics of neural crest stem cells, at a scale bar of 50 μm, where FIG. 3A is a result of EGFP, SOX10, and DAPI co-staining in a control group; FIG. 3B is a result of EGFP, SOX10, and DAPI co-staining in a silicosis group; and FIG. 3C is a partial magnification of FIG. 3B;

FIG. 4A to FIG. 4C show that a group of EGFP-positive cells in fibrotic lesions of the mT/mG;Wnt1-Cre transgenic mouse exhibit characteristics of mesenchymal cells, at a scale bar of 50 μm, where FIG. 4A is a result of EGFP, ACTA2, and DAPI co-staining in the control group; FIG. 4B is a result of EGFP, ACTA2, and DAPI co-staining in the silicosis group; and FIG. 4C is a partial enlargement of FIG. 4B;

FIG. 5 shows flow cytometric detection results of a proportion of the neural crest derived cell from peripheral blood of the mT/mG;Wnt1-Cre transgenic mouse;

FIG. 6A to FIG. 6C show microscopic examination photos of a peripheral blood mononuclear cell of the mT/mG;Wnt1-Cre transgenic mouse on the 1st day of culture (200×), where arrows indicate EGFP-positive cells (the same below), where FIG. 6A is a bright field image under a microscope; FIG. 6B is an immunofluorescence staining image of EGFP antibody; and FIG. 6C is a superimposed image of FIG. 6A and FIG. 6B;

FIG. 7A to FIG. 7C show microscopic examination photos of a peripheral blood mononuclear cell of the mT/mG;Wnt1-Cre transgenic mouse on the 3rd day of culture (200×), where FIG. 7A is a bright field image under a microscope; FIG. 7B is an immunofluorescence staining image of EGFP antibody; and FIG. 7C is a superimposed image of FIG. 7A and FIG. 7B;

FIG. 8A to FIG. 8C show microscopic examination photos of a peripheral blood mononuclear cell of the mT/mG;Wnt1-Cre transgenic mouse on the 9th day of culture (200×), where FIG. 8A is a bright field image under a microscope; FIG. 8B is an immunofluorescence staining image of EGFP antibody; and FIG. 8C is a superimposed image of FIG. 8A and FIG. 8B;

FIG. 9A to FIG. 9C show microscopic examination photos of a peripheral blood mononuclear cell of the mT/mG;Wnt1-Cre transgenic mouse on the 14th day of culture (100×), where FIG. 9A is a bright field image under a microscope; FIG. 9B is an immunofluorescence staining image of EGFP antibody; and FIG. 9C is a superimposed image of FIG. 9A and FIG. 9B;

FIG. 10A to FIG. 10C show microscopic examination photos of a peripheral blood mononuclear cell of the mT/mG;Wnt1-Cre transgenic mouse on the 1st day of first subculture (100×), where FIG. 10A is a bright field image under a microscope; FIG. 10B is an immunofluorescence staining image of EGFP antibody; and FIG. 10C is a superimposed image of FIG. 10A and FIG. 10B;

FIG. 11A to FIG. 11C show microscopic examination photos of a peripheral blood mononuclear cell of the mT/mG;Wnt1-Cre transgenic mouse on the 3rd day of first subculture (100×), where FIG. 11A is a bright field image under a microscope; FIG. 11B is an immunofluorescence staining image of EGFP antibody; and FIG. 11C is a superimposed image of FIG. 11A and FIG. 11B; and

FIG. 12A to FIG. 12C show microscopic examination photos of a peripheral blood mononuclear cell of the mT/mG;Wnt1-Cre transgenic mouse on the 5th day of first subculture (100×), where FIG. 12A is a bright field image under a microscope; FIG. 12B is an immunofluorescence staining image of EGFP antibody; and FIG. 12C is a superimposed image of FIG. 12A and FIG. 12B.

### DETAILED DESCRIPTION OF THE EMBODIMENTS

mT/mG;Wnt1-Cre Mice:

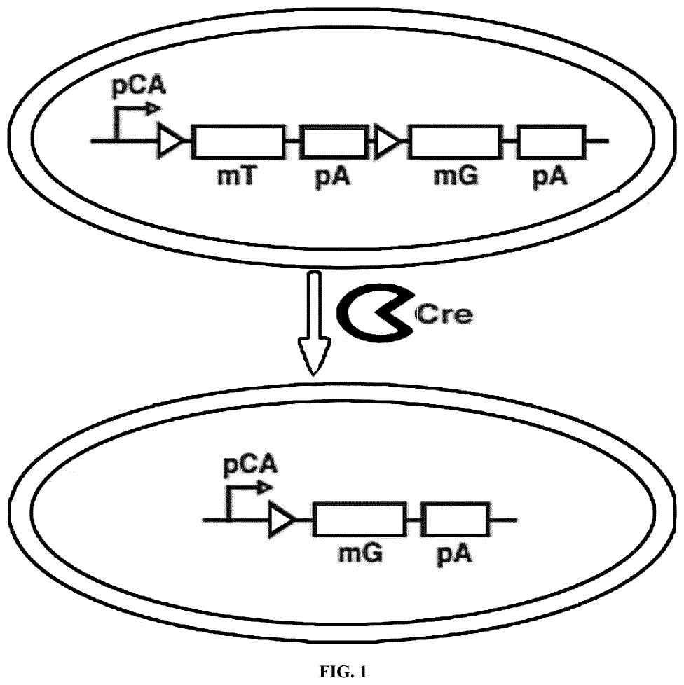

The Wnt1-Cre transgenic mouse (129S4.Cg-E2f1 Tg(Wnt1-Cre)2Sor/J; Stock No: 022137) and the mT/mG double-fluorescent reporter mouse (B6.129(Cg)-Gt(ROSA)26Sor tm4 (ACTB-tdTomato-EGFP)Luo/J; Stock No: 007676) both are purchased from Jackson Laboratory. The Wnt1-Cre transgenic mouse line is widely used to study neural crest and its derivatives as well as the development of midbrain, and is currently the most common tool mouse line for studying neural crest derived cells. Before Cre recombination in the mT/mG double-fluorescent reporter mouse, the fluorescent expression of tdTomato (mT) localized on the cell membrane spreads throughout the cells/tissues. Cells expressing Cre recombinase (and cell lineages derived from these cells) have fluorescent expression of EGFP (mG) that is localized to the cell membrane, emitting green fluorescence instead of red fluorescence. Therefore, when 8-12-week-old Wnt1-Cre transgenic mouse is crossed with mT/mG double-fluorescent reporter mouse to obtain the mT/mG;Wnt1-Cre mouse as offspring, the cell membrane surface of cells or tissues derived from neural crest can exhibit green fluorescence. After crossing the Wnt1-Cre transgenic mouse with the mT/mG double-fluorescent reporter mouse, four genetic models of offspring mice can be obtained, including a wild-type mouse, a Wnt1-Cre transgenic mouse, an mT/mG double-fluorescent reporter mouse, and an mT/mG;Wnt1-Cre mouse, where the mT/mG;Wnt1-Cre mouse shows a birth probability of about 20%. Therefore, it is necessary to extract a DNA from offspring mice to allow genotype identification and screening. FIG. 1 shows a schematic view for a generation principle of the mT/mG;Wnt1-Cre transgenic mouse.

### Example 1

The Wnt1-Cre transgenic mouse (129S4.Cg-E2f1 Tg(Wnt1-Cre)2Sor/J; Stock No: 022137) and the mT/mG double-fluorescent reporter mouse (B6.129(Cg)-Gt(ROSA)26Sor tm4 (ACTB-tdTomato-EGFP)Luo/J; Stock No: 007676) both were purchased from Jackson Laboratory. After mating, the mT/mG;Wnt1-Cre transgenic mouse was selected through genetic identification by a Southern method. EGFP-positive cells in this mouse were considered to be neural crest derived cells.

1. Genotype Identification of the mT/mG;Wnt1-Cre Mice

1.1. Extraction of Mouse DNA
(1) Toes of the mouse were cut and numbered. The toes of the mouse were collected, or about 0.5 cm of a tail section was cut from the end of its tail, placed in a 1.5 mL sterile EP tube, and stored at −20° C.
(2) 200 μL of non-SDS buffer+5 μL of proteinase K were added to each centrifuge tube containing the tissue, and incubated overnight in a 55° C. water bath.
(3) The next day, the centrifuge tube in the water bath was vortexed for 5 s, and then centrifuged at 13,000 rpm for 1 min.
(4) The proteinase K was activated by heating in a 98° C. water bath for 10 min.
(5) Centrifugation was conducted at 13,000 rpm for 1 min, and 100 μL of a resulting supernatant was collected.
(6) The supernatant was stored at −20° C.

1.2. PCR System

Wnt1-Cre primer design, 5′→3′:
Transgene Forward 16773:
(SEQ ID NO: 1)
CAG CGC CGC AAC TAT AAG AG,
Transgene Reverse16774:
(SEQ ID NO: 2)
CAT CGA CCG GTA ATG CAG,
Internal Positive Control8744:
(SEQ ID NO: 3)
CAA ATG TTG CTT GTC TGG TG,
Internal Positive Control8745:
(SEQ ID NO: 4)
GTC AGT CGA GTG CAC AGT TT.
EGFP primer design, 5′→3′:
Common12177:
(SEQ ID NO: 5)
CTT TAA GCC TGC CCA GAA GA,
Mutant Forward30297:
(SEQ ID NO: 6)
TAG AGC TTG CGG AAC CCT TC,
Wild type Forward30298:
(SEQ ID NO: 7)
AGG GAG CTG CAG TGG AGT AG,

A total volume of Wnt1-cre gene and EGFP genotype reaction systems was 25 μL.

A Wnt1-cre gene PCR reaction system included the following components:

template DNA, 1 μL;

2-Hieff HotStart PCR Genotyping Master Mix(with Dye), 12.5 μL;

Transgene Forward 16773, 1 μL;

Transgene Reverse 16774, 1 μL;

Internal Positive Control8744, 1 μL;

Internal Positive Control8745, 1 μL;

ddH20, making up to 25 μL.

An EGFP gene PCR reaction system included the following components:

template DNA, 1 μL;

2-Hieff HotStart PCR Genotyping Master Mix(with Dye), 12.5 μL;

Common12177, 1 μL;

Mutant Forward 30297, 1 μL;

Wild type Forward 30298, 1 μL;

ddH20, making up to 25 μL.

1.3. PCR Reaction Conditions

The reaction conditions for Wnt1-cre gene and EGFP genotype were as follows:
(1) initial denaturation at 95° C. for 5 min;
(2) denaturation at 95° C. for 30 s, Wnt1-cre gene annealing at 60° C. for 30 s/EGFP gene annealing at 65° C. for 30 s, and extension at 72° C. for 30 s, repeating for 35 cycles; and
(3) final extension at 72° C. for 10 min.
(4) PCR amplification products of the Wnt1-cre gene and EGFP gene and 100 bp DNA Marker were selected to allow 1.5% agarose gel electrophoresis at 100 U for 50 min.

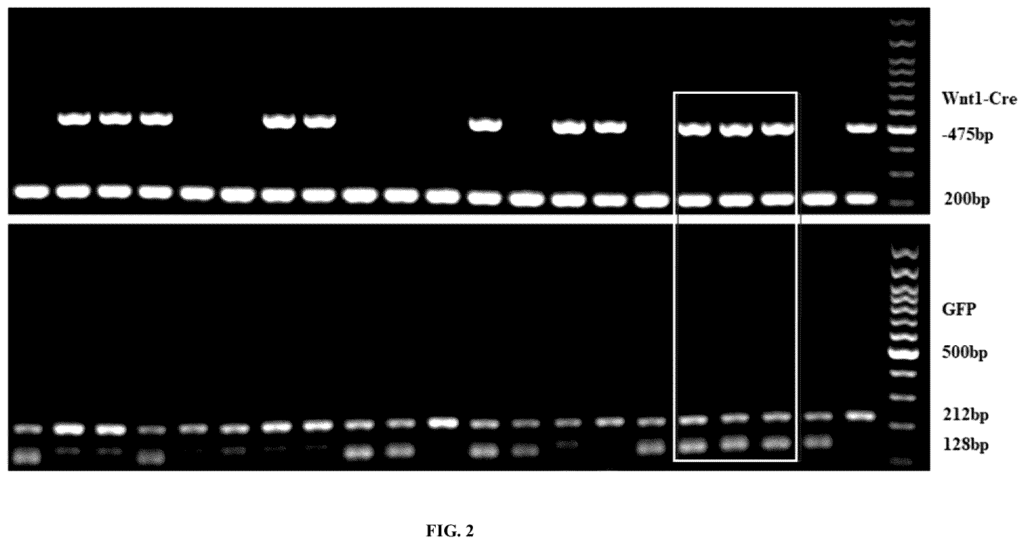

In the present disclosure, the genotype detection of the mT/mG;Wnt1-Cre transgenic mouse was shown in FIG. 2. This mouse showed distinct four bands ranging in size of 200 bp-475 bp and 128 bp-212 bp. The wild-type mice showed two bands of 200 bp and 212 bp. The Wnt1-Cre mouse showed three bands of 200 bp-475 bp and 212 bp, while the mT/mG mouse showed three bands of 200 bp and 128 bp-212 bp or two bands of 200 bp and 128 bp.

### Example 2

The mT/mG;Wnt1-Cre mice at 8 to 12 weeks were randomly divided into a physiological saline group and a SiO₂ model group. 1 d before modeling, an appropriate amount of SiO₂ dust was heated in a clean petri dish at 160° C. for 2 h, cooled slightly and then ground in an agate mortar for 30 min. An obtained powder was mixed with sterile physiological saline to form a 25 mg/mL suspension for later use. The mice were anesthetized by intraperitoneal injection of 4% chloral hydrate at a dose of 0.01 mL/g. After 3 min to 4 min, when the mouse's back-and-forth reflex and toe-pinching reflex disappeared, the mouse's abdomen was placed downward, with its upper incisors suspended on a thin line on an operating table, and its neck was illuminated with a cold light source. The mouse's tongue was pulled out with small tweezers of left hand, the mouse's upper jaw was held using one end of small straight tweezers on right hand, with other end against the base of the mouse's tongue. The field of view in mouse's mouth was exposed as possible to observe the position of vocal cords by assistance of the cold light source. When seeing the trachea being translucent, a small bright spot could be seen if watching closely, which was the glottis. When the small bright spot became larger (that is, when the glottis was most opened), a 22G indwelling needle was promptly inserted into the trachea, the needle core was withdrawn, a 1 mL syringe was inserted into the cap of the 22G indwelling needle, tightened by rotating, such that tracheal intubation was completed. The mice in the model group were perfused with 0.1 mL of a silica suspension through the trachea via the oral cavity according to the above method, while the mice in the physiological saline group were perfused with an equal volume of 0.1 mL of sterile physiological saline. The mice were made stand upright and gently shaken from side to side to help the SiO₂ suspension distribute evenly while avoiding suffocation of the mouse's organs due to the dust. On the 30th day after SiO₂ treatment, mouse lung tissues were fixated with 4% (mass percentage) paraformaldehyde, dehydrated with 30% (mass percentage) sucrose solution, embedded by OCT, and then cut into 6 m frozen sections to allow immunofluorescence detection. The frozen sections were permeabilized with 0.5% Triton X-100, blocked with 5% BSA at 37° C. for 30 min, incubated with primary antibodies such as SOX10 (1:50, abcam) and Acta2 (1:500, CST) at 4° C. for 12 h, incubated with fluorescent secondary antibody Alexa Flour647 (1:1000, abcam) at room temperature for 1 h, stained with DAPI (Beyotime) on the nuclei for 2 min, and then washed with PBS to remove excess DAPI. After sealing with anti-fluorescence quenching mounting solution, the slides were observed under a Zeiss LSM880 ultra-high resolution inverted confocal microscope.

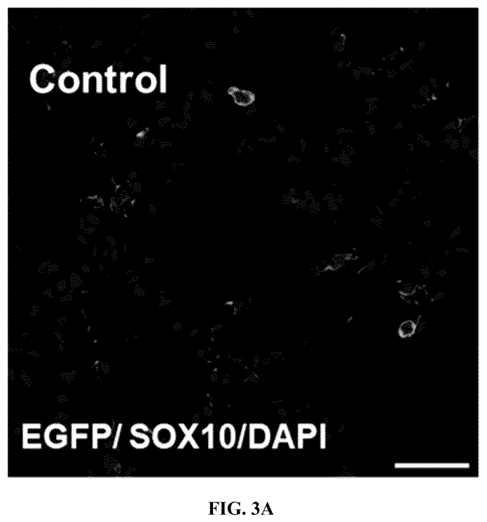

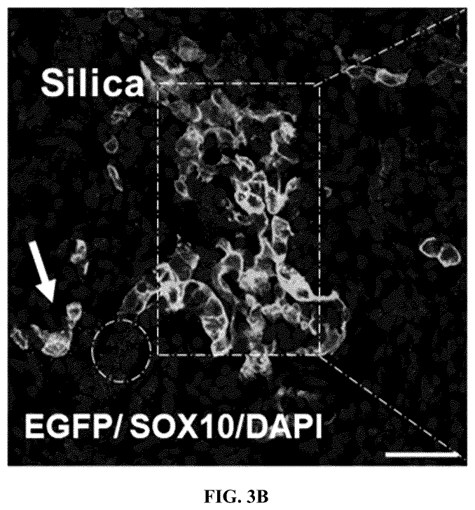

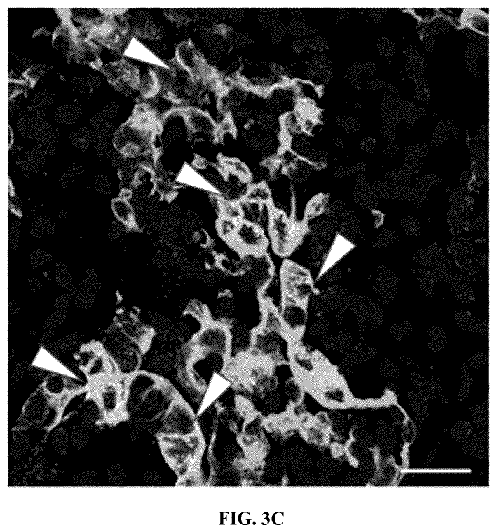

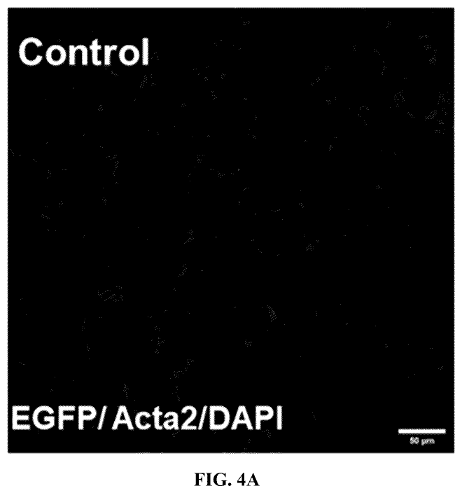

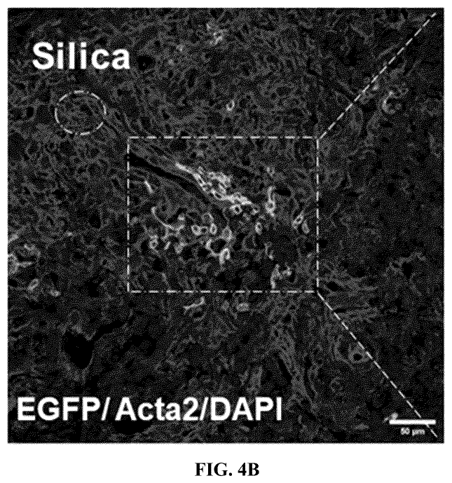

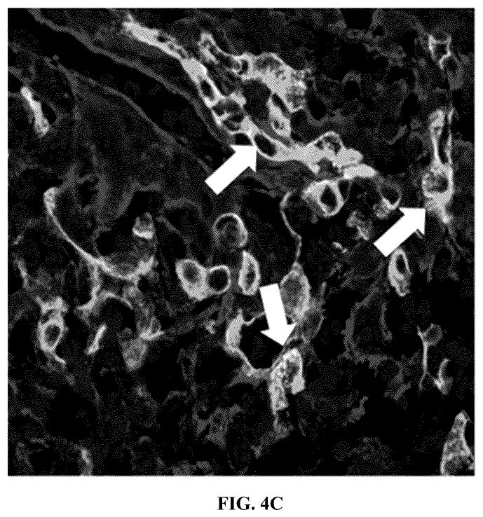

An abnormally aggregated population of neural crest derived cells was found in silica-induced silicosis fibrosis in mT/mG;Wnt1-Cre mouse. This cell population not only had the characteristics of neural crest stem cells, but also had the characteristics of mesenchymal cells, as shown in FIG. 3A to FIG. 3C and FIG. 4A to FIG. 4C.

FIG. 3A to FIG. 3C showed that the group of EGFP-positive cells in fibrotic lesions of the mT/mG;Wnt1-Cre transgenic mouse exhibited characteristics of neural crest stem cells, where the long arrow in the middle picture indicated EGFP⁺ cells, which were green under the fluorescence image; circles represented SOX10⁺ cells, which were magenta under the fluorescence image; the box indicated the EGFP and SOX10 double-positive cell population; the arrow in the rightmost image represented a magnification effect of the double-positive cell population, photographed under a 200× microscope at a scale bar of 50 μm.

FIG. 4A to FIG. 4C showed that the group of EGFP-positive cells in fibrotic lesions of the mT/mG;Wnt1-Cre transgenic mouse exhibited characteristics of mesenchymal cells, where the circle in the middle represented Acta2⁺ cells, which were magenta under the fluorescence image; the box indicated the EGFP and Acta2 double-positive cell population; the arrow in the rightmost image represented a magnification effect of the double-positive cell population, photographed under a 200× microscope at a scale bar of 50 μm.

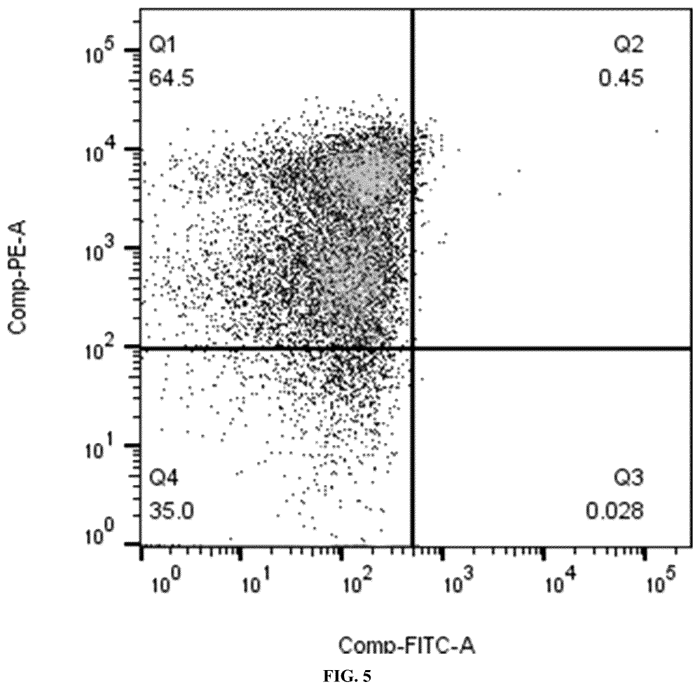

This discovery provided a new idea for exploring the mechanism of neural crest derived cells in silicosis fibrosis. At the same time, flow cytometry analysis was conducted to find the presence of EGFP-positive cells in the peripheral blood of mice, that is, neural crest derived cells (FIG. 5).

In order to better and in-depth study the role of this cell population in silicosis fibrosis, EGFP-positive cells were planned to be extracted from the peripheral blood of mT/mG;Wnt1-Cre mouse for in vitro culture. This method had not been reported yet.

### Example 3

The neural crest derived cells were separated from the mT/mG;Wnt1-Cre transgenic mouse, including the following steps:
(1) Blood was collected from orbital vein to obtain about 1 mL of anticoagulated blood as the peripheral blood, and the peripheral blood was diluted with a PBS solution at a volume ratio of 1:1.
(2) Obtained diluted peripheral blood was slowly added onto a liquid surface of about 3 mL of a mouse peripheral blood mononuclear cell separation medium (Tianjin Haoyang TBD, mouse peripheral blood mononuclear cell separation medium).
(3) Centrifuging was conducted at 400 g and 20° C. for 30 min.
(4) At least 800 μL of the buffy coat cell was collected and the PBS solution was added to 14 mL.
(5) Centrifuging was conducted at 300 g for 10 min.
(6) A resulting supernatant was discarded, 3 mL of the red blood cell lysis solution was added, the remaining red blood cell was mixed evenly by gently pipetting, and the red blood cell was lysed at a room temperature for 2 min.
(7) Centrifuging was conducted at 400 g and 4° C. for 5 min.
(8) A resulting red supernatant was discarded, and 10 mL of the PBS solution was added to mix a remaining cell evenly by gently pipetting.
(9) Centrifugation was conducted at 400 g for 3 min; a resulting supernatant was discarded.
(10) Optional step: 10 mL of the PBS solution was added, and a remaining cell was mixed evenly by gently pipetting; centrifuging was conducted at 400 g for 3 min; a resulting supernatant was discarded.
(11) The cell was resuspended in a newly configured DMEM/F12 medium and cell counting was conducted.
(12) The cell was cultured in vitro: the DMEM/F12 medium was changed with a new medium three days after the cell was inoculated, semi-quantitative medium change was conducted every 3 d to 4 d for first two weeks, digestion and subculture were conducted when a cell confluence reached about 80%, and the medium was changed once a week for six weeks to obtain the neural crest derived cell.

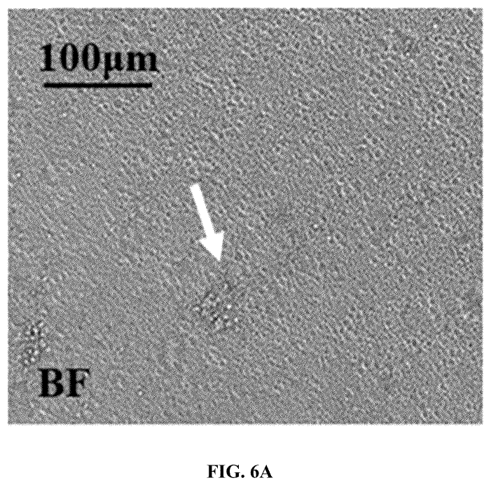

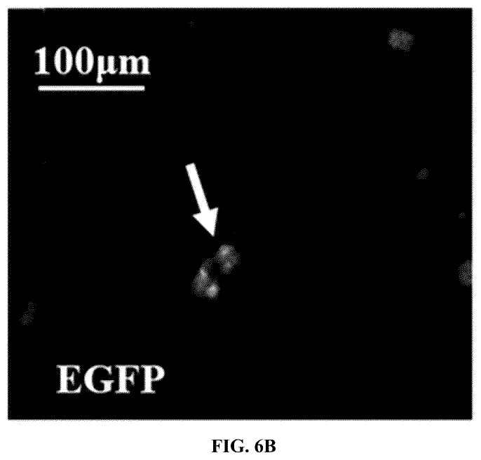

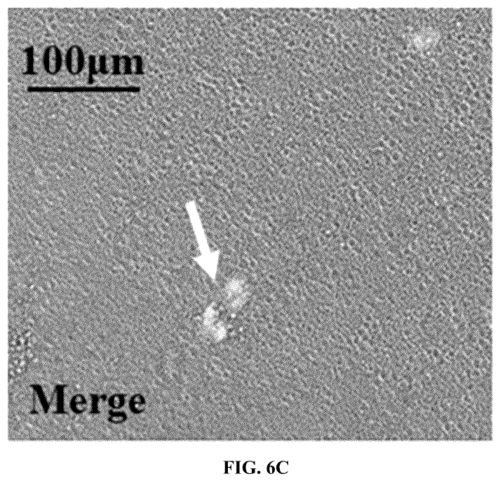

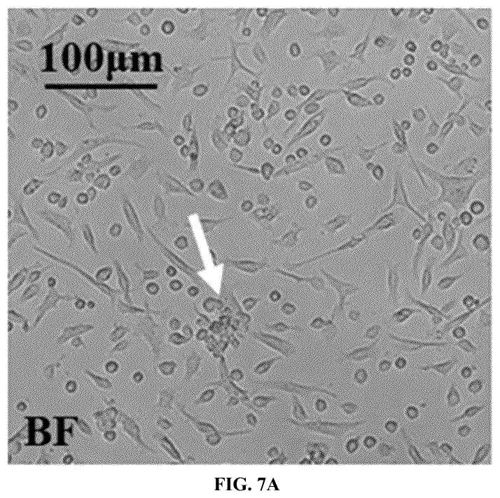

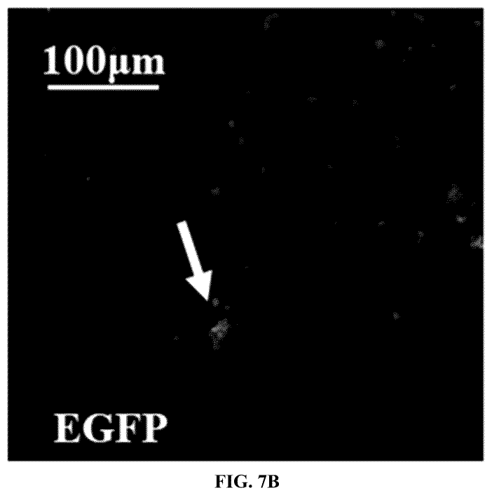

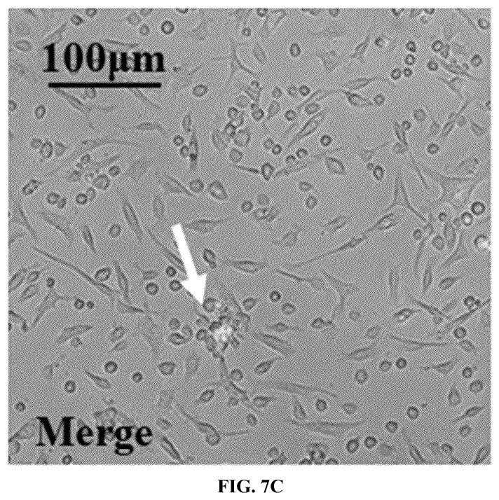

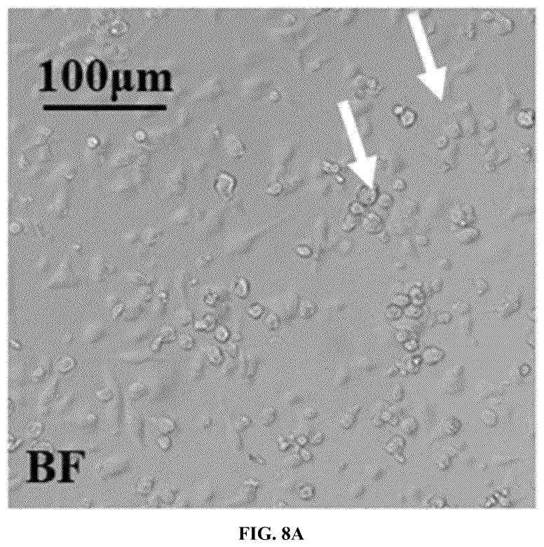

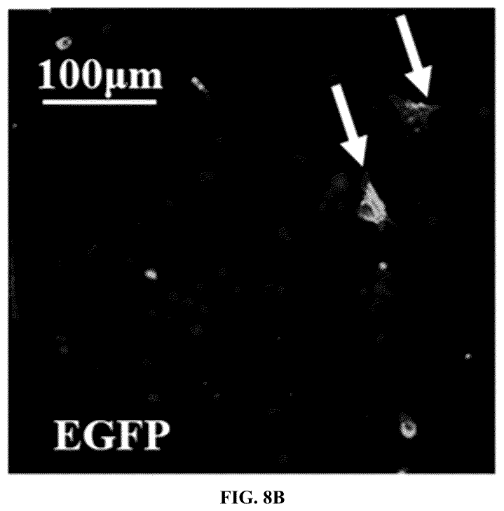

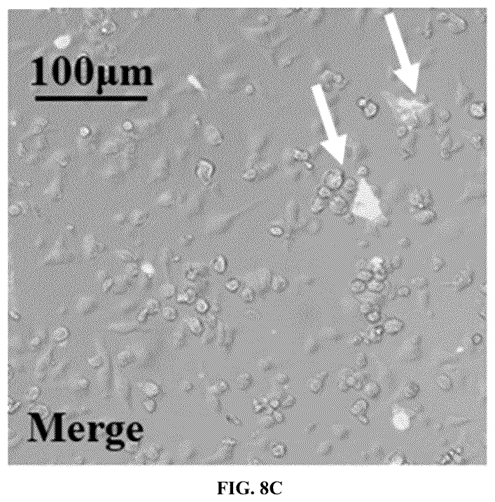

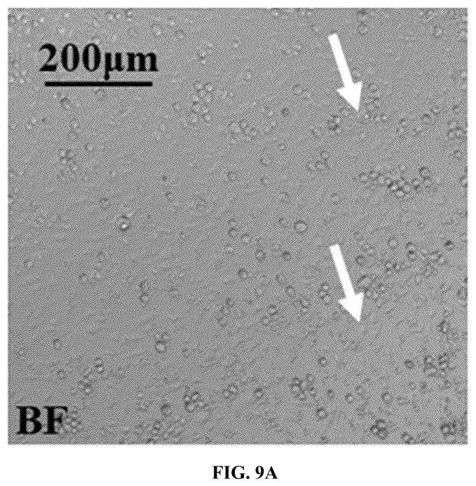

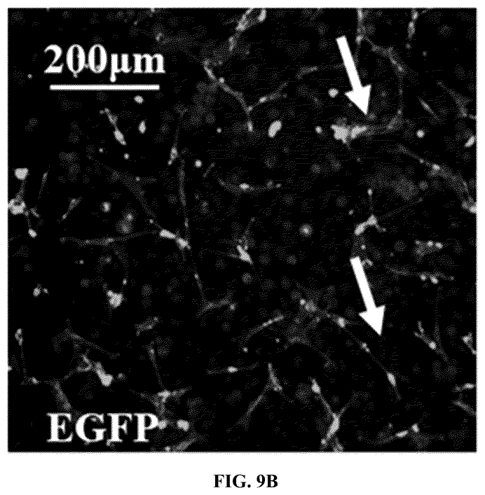

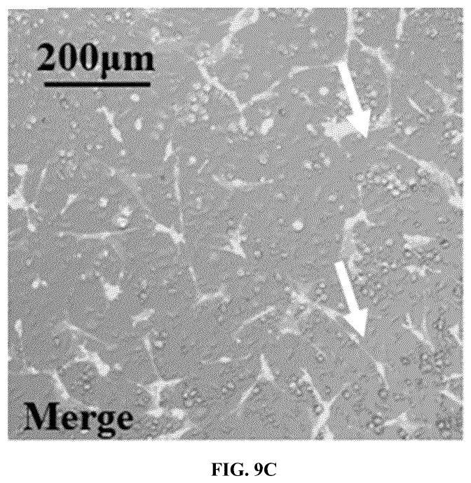

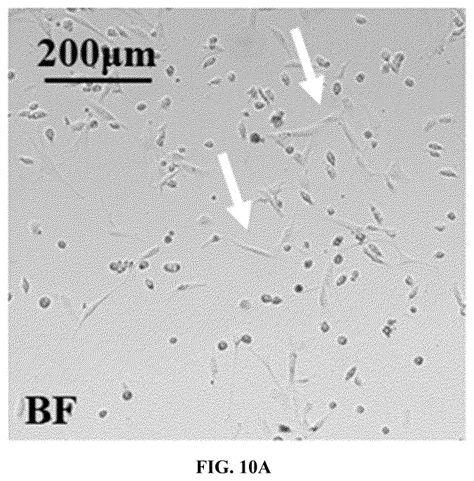

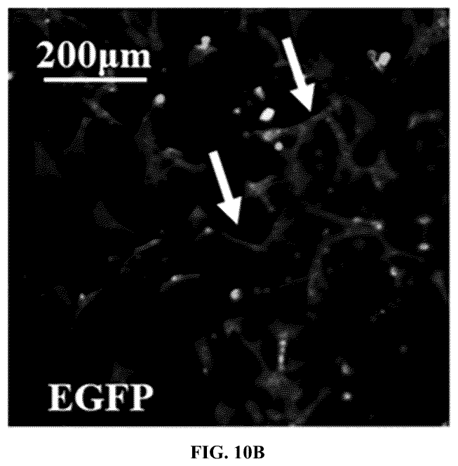

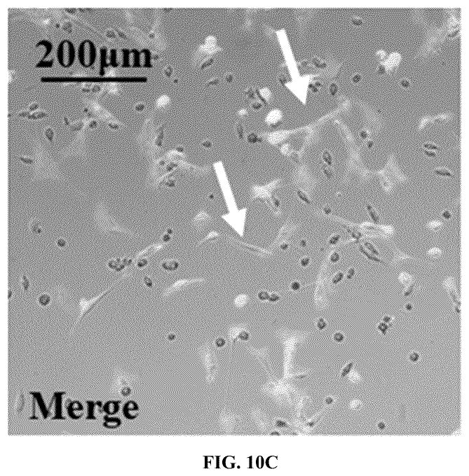

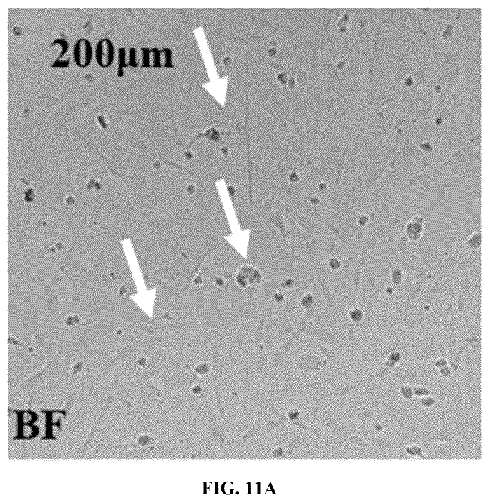

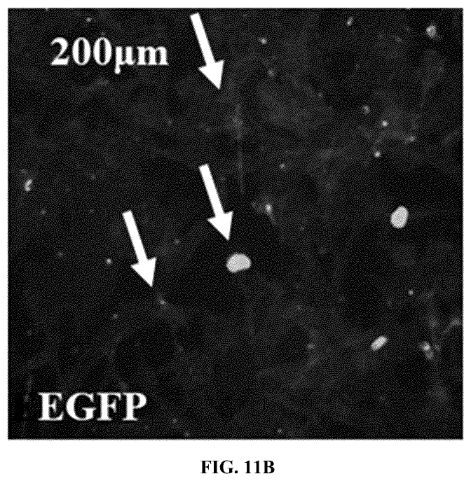

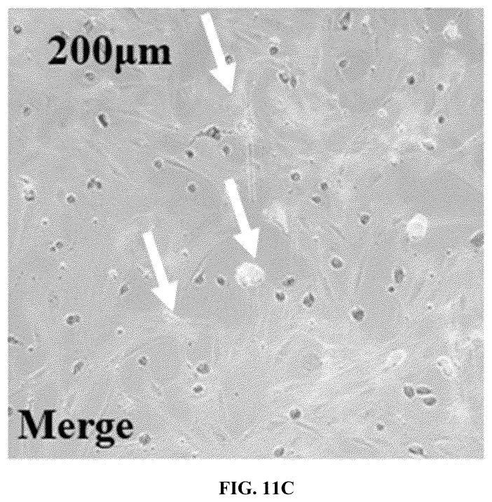

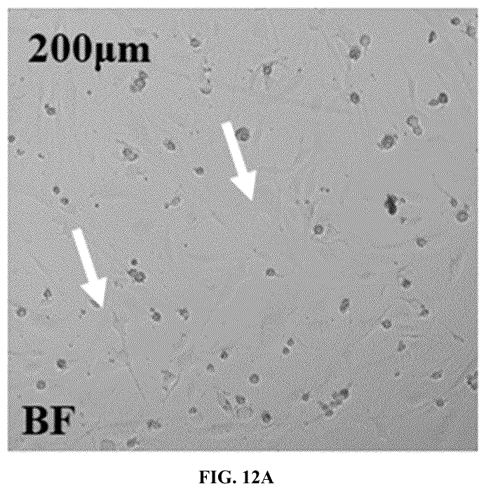

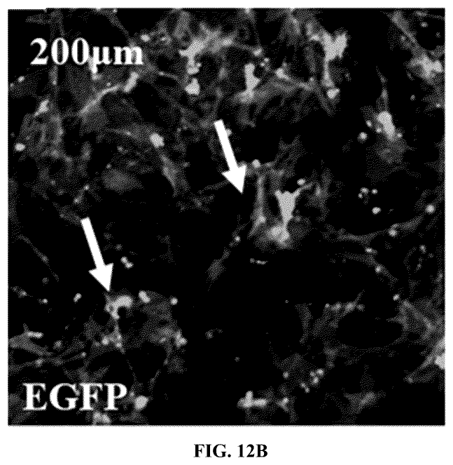

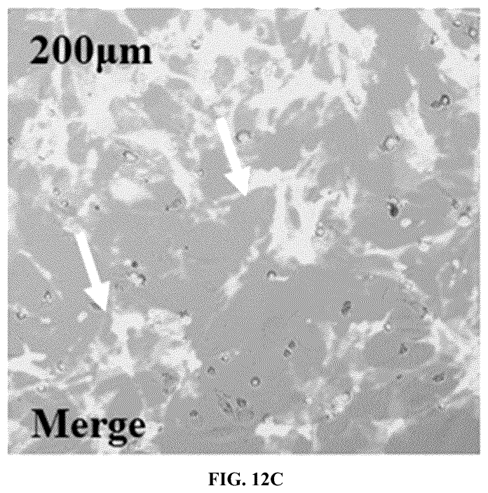

The separated neural crest derived cells were observed. FIG. 6A to FIG. 6C, FIG. 7A to FIG. 7C, FIG. 8A to FIG. 8C, and FIG. 9A to FIG. 9C showed microscopic observation results of the peripheral blood mononuclear cell of the mT/mG;Wnt1-Cre transgenic mouse on the 1st, 3rd, 9th, and 14th days of culture, respectively. Characteristic results of neural crest derived cells from peripheral blood showed: the cell morphology observation under a phase contrast microscope indicated that at the initial stage of mononuclear cell inoculation, most of adherent cells were round; 3 days after inoculation, non-adherent suspended cells were removed, and the medium was replaced with a new medium; on the 3rd day of culture, the adherent cells grew in clusters. The cells growing in aggregates were observed under an inverted fluorescence microscope to be EGFP-positive, indicating that these cells were neural crest derived cells.

What is claimed is:

1. A method for separating a neural crest derived cell from peripheral blood, comprising separating the neural crest derived cell from peripheral blood of a subject to be separated.

2. The method for separating a neural crest derived cell from peripheral blood according to claim 1, wherein the subject to be separated is an experimental animal.

3. The method for separating a neural crest derived cell from peripheral blood according to claim 2, wherein the experimental animal is selected from the group consisting of an experimental mouse, an experimental rat, and an experimental monkey.

4. The method for separating a neural crest derived cell from peripheral blood according to claim 2, specifically comprising the following steps:
(1) collecting the peripheral blood and separating a buffy coat cell using a peripheral blood mononuclear cell separation medium;
(2) adding a red blood cell lysis solution to lyse a red blood cell; and
(3) centrifuging to obtain a peripheral blood mononuclear cell without red blood cells, wherein the neural crest derived cell is mixed in the peripheral blood mononuclear cell without red blood cells.

5. The method for separating a neural crest derived cell from peripheral blood according to claim 4, wherein the peripheral blood is collected from an orbital vein when the experimental animal is the experimental mouse.

6. The method for separating a neural crest derived cell from peripheral blood according to claim 4, wherein an mT/mG;Wnt1-Cre mouse obtained by hybridization screening of a Wnt1-Cre transgenic mouse and an mT/mG double-fluorescent reporter mouse is used when the experimental animal is the experimental mouse.

7. The method for separating a neural crest derived cell from peripheral blood according to claim 6, wherein the peripheral blood is collected to separate the neural crest derived cell after silicosis fibrosis is induced in the mT/mG;Wnt1-Cre mouse by silica.

8. The method for separating a neural crest derived cell from peripheral blood according to claim 4, specifically comprising the following steps:
(1) collecting 1 mL of anticoagulated blood as the peripheral blood, and diluting the peripheral blood with a PBS solution at a volume ratio of 1:1;
(2) slowly adding obtained diluted peripheral blood onto a liquid surface of 3 mL of a mouse peripheral blood mononuclear cell separation medium;
(3) centrifuging at 400 g and 20° C. for 30 min;
(4) collecting at least 800 μL of the buffy coat cell and adding the PBS solution to 14 mL;
(5) centrifuging at 300 g for 10 min;
(6) discarding a resulting supernatant, adding 3 mL of the red blood cell lysis solution, mixing the remaining red blood cell evenly by gently pipetting, and lysing the red blood cell at a room temperature for 2 min;
(7) centrifuging at 400 g and 4° C. for 5 min;
(8) discarding a resulting red supernatant, and adding 10 mL of the PBS solution, and mixing a remaining cell evenly by gently pipetting;
(9) centrifuging at 400 g for 3 min, and discarding a resulting supernatant;
(10) optional step: adding 10 mL of the PBS solution, and mixing a remaining cell evenly by gently pipetting; centrifuging at 400 g for 3 min, and discarding a resulting supernatant;
(11) resuspending the cell in a newly configured DMEM/F12 medium and conducting cell counting; and
(12) culturing the cell in vitro: changing the DMEM/F12 medium with a new medium three days after the cell is inoculated, conducting semi-quantitative medium change every 3 d to 4 d for first two weeks, conducting digestion and subculture when a cell confluence reaches 80%, and changing the medium once a week for six weeks to obtain the neural crest derived cell.


SEQUENCE LISTING (7 sequences)

SEQ ID NO: 1 - DNA, 20 bp, synthetic construct
  source 1..20: mol_type = other DNA; note = Transgene Forward 16773
cagcgccgcaactataagag

SEQ ID NO: 2 - DNA, 18 bp, synthetic construct
  source 1..18: mol_type = other DNA; note = Transgene Reverse16774
catcgaccggtaatgcag

SEQ ID NO: 3 - DNA, 20 bp, synthetic construct
  source 1..20: mol_type = other DNA; note = Internal Positive Control8744
caaatgttgcttgtctggtg

SEQ ID NO: 4 - DNA, 20 bp, synthetic construct
  source 1..20: mol_type = other DNA; note = Internal Positive Control8745
gtcagtcgagtgcacagttt

SEQ ID NO: 5 - DNA, 20 bp, synthetic construct
  source 1..20: mol_type = other DNA; note = Common12177
ctttaagcctgcccagaaga

SEQ ID NO: 6 - DNA, 20 bp, synthetic construct
  source 1..20: mol_type = other DNA; note = Mutant Forward30297
tagagcttgcggaacccttc

SEQ ID NO: 7 - DNA, 20 bp, synthetic construct
  source 1..20: mol_type = other DNA; note = Wild type Forward30298
agggagctgcagtggagtag